(19) United States
(12) Patent Application Publication
(10) Pub. No.: US 2024/0293333 A1
(43) Pub. Date: Sep. 5, 2024

(54) TOPICAL DELIVERY OF LIPOPROTEIN-MIMETIC NANOPARTICLES

(71) Applicant: NORTHWESTERN UNIVERSITY, Evanston, IL (US)

(72) Inventors: Timothy Feliciano, Chicago, IL (US); Stephen E. Henrich, Chicago, IL (US); C. Shad Thaxton, Chicago, IL (US); Robert M. Lavker, Evanston, IL (US); Kurt Lu, Evanston, IL (US); Han Peng, Evanston, IL (US); Nihal Kaplan, Evanston, IL (US); Ummiye Venus Onay, Evanston, IL (US)

(21) Appl. No.: 18/280,915

(22) PCT Filed: Mar. 11, 2022
(86) PCT No.: PCT/US22/19884

§ 371 Date: Sep. 7, 2023

Related U.S. Application Data
(60) Provisional application No. 63/160,713, filed on Mar. 12, 2021.

(51) Int. Cl. A61K 9/51; A61K 9/00; A61K 31/593; A61P 29/00

(52) U.S. Cl. CPC A61K 9/5123; A61K 9/0048; A61K 31/593; A61P 29/00

(57) ABSTRACT

Disclosed herein are compositions comprising synthetic nanoparticles and methods of use thereof for reducing inflammation of the eye and/or skin.

### RELATED APPLICATION

This application claims the benefit under 35 U.S.C. § 119(e) of U.S. provisional application Ser. No. 63/160,713, filed Mar. 12, 2021, the entire contents of which is incorporated by reference herein.

### BACKGROUND OF THE INVENTION

The active form of vitamin D3 (1,25 dihydroxyvitamin D3), is a potent suppressor of inflammation in the eye and skin. For instance, topical administration of vitamin D3 reduces corneal infiltration of immune cells, reduces corneal neovascularization, and reduces pro-inflammatory cytokines in the cornea. Moreover, topical delivery of vitamin D3 is widely used for treatment of psoriasis. In the skin, vitamin D3 is known to induce thymic stromal lymphopoietin and cathelicidin which are potent regulators of Th2 immunity and innate immunity respectively. Vitamin D3-mediated induction of these factors leads to suppression of IL-1 alpha, IL-1 beta, TNF-alpha and other pro-inflammatory cytokines, which ultimately improves the psoriatic condition. However, most current formulations of vitamin D3 lack a sophisticated carrier, which is problematic because Vitamin D3 is ordinarily transported in the body by lipoproteins and other fat carriers. Accordingly, novel methods and compositions for the delivery of Vitamin D3 and related anti-inflammatory molecules are provided.

### SUMMARY OF THE INVENTION

The present disclosure is based, at least in part, on compositions, kits, and methods for reducing inflammation in the eye or skin of a subject by administering a synthetic nanoparticle (e.g., a high density lipoprotein nanoparticle (HDL-NP)).

Accordingly, some aspects of the present disclosure provide a method of reducing inflammation in the eye or skin of a subject. In some embodiments, a method of reducing inflammation in the eye or skin of a subject comprises administering to the eye or skin of subject a synthetic nanoparticle comprising a nanoparticle core and a shell comprising a lipid, wherein the shell is surrounding and attached to the nanoparticle core, wherein the synthetic nanoparticle is administered in an effective amount to reduce inflammation in the eye or skin.

In some embodiments, the synthetic nanoparticle further comprises an anti-inflammatory molecule. In some embodiments, the anti-inflammatory molecule is Vitamin D3. In some embodiments, the anti-inflammatory molecule is associated with the core of the synthetic nanoparticle and/or the shell of the synthetic nanoparticle. In some embodiments, the anti-inflammatory molecule is attached to the synthetic nanoparticle via a covalent bond, ionic interactions, hydrophobic and/or hydrophilic interactions, electrostatic interactions, van der Waals interactions, or combinations thereof. In some embodiments, the nanoparticle comprises 10-100 anti-inflammatory molecules, optionally wherein the nanoparticle comprises about 40 anti-inflammatory molecules.

Some aspects of the present disclosure provides a method of delivering an anti-inflammatory molecule to the eye or skin of a subject. In some embodiments, the method of delivering an anti-inflammatory molecule to the eye or skin of a subject comprises administering to the eye or skin of the subject a synthetic nanoparticle comprising a nanoparticle core and a shell comprising a lipid, wherein the shell is surrounding and attached to the nanoparticle core, wherein the synthetic nanoparticle further comprises an anti-inflammatory molecule.

In some embodiments, the anti-inflammatory molecule is Vitamin D3. In some embodiments, the anti-inflammatory molecule is associated with the core of the synthetic nanoparticle and/or the shell of the synthetic nanoparticle. In some embodiments, the anti-inflammatory molecule is attached to the synthetic nanoparticle via a covalent bond, ionic interactions, hydrophobic and/or hydrophilic interactions, electrostatic interactions, van der Waals interactions, or combinations thereof. In some embodiments, the nanoparticle comprises 10-100 anti-inflammatory molecules, optionally wherein the nanoparticle comprises about 40 anti-inflammatory molecules.

Some aspects of the present disclosure provide a method of reducing inflammation in the eye or skin of a subject comprising administering to the eye or skin of the subject an anti-inflammatory formulation comprising (a) a synthetic nanoparticle comprising a nanoparticle core and a shell comprising a lipid, wherein the shell is surrounding and attached to the nanoparticle core, and (b) an anti-inflammatory molecule; wherein the anti-inflammatory formulation is administered in an effective amount to reduce inflammation in the eye or skin.

In some embodiments, the anti-inflammatory molecule is Vitamin D3.

In some embodiments, the administering step comprises topical administration, intraocular administration, or intradermal administration.

In some embodiments, the method results in reduction of expression of at least one inflammatory gene in the eye or skin relative to a baseline measurement. In some embodiments, the at least one inflammatory gene is selected from the group consisting of: Acta2, Enos, Il1a, Inos, Tgfb, Il12r, Pdgfb, Vegfa, Cox2, Il1b, Il6, Mmp12, Mmp9, and Ccl2. In some embodiments, the baseline measurement is an expression level of the eye or skin prior to the administering step, or an expression level of an untreated eye or skin.

In some embodiments, the subject is a human subject. In some embodiments, the subject has an inflammatory disease or disorder. In some embodiments, the inflammatory disease or disorder is selected from the group consisting of: corneal inflammation, corneal regeneration, ocular inflammation, age-related skin deterioration, psoriasis, atopic dermatitis, and ocular surface diseases. In some embodiments, the ocular surface diseases are selected from the group consisting of: chemical and thermal injury, long-term contact lens wear, severe chronic rosacea, Stevens-Johnson syndrome, atopic keratoconjunctivitis, bacterial keratitis and graft versus host disease.

In some embodiments, the method comprises administering the synthetic nanoparticle or anti-inflammatory formulation at least once per day, at least once per week, or at least once per month. In some embodiments, the method comprises administering the synthetic nanoparticle or anti-inflammatory formulation twice per day for three days.

Some aspects of the present disclosure provide a synthetic nanoparticle comprising a nanoparticle core, a shell comprising a lipid surrounding and attached to the nanoparticle core, and an anti-inflammatory molecule. In some embodiments, the anti-inflammatory molecule is Vitamin D3. In some embodiments, the anti-inflammatory molecule is attached to the synthetic nanoparticle via a covalent bond, ionic interactions, hydrophobic and/or hydrophilic interactions, electrostatic interactions, van der Waals interactions, or combinations thereof.

In some embodiments, the core is an inorganic core, optionally wherein the inorganic core is comprised of gold (Au).

In some embodiments, the synthetic nanoparticle further comprises a protein. In some embodiments, the protein is an apolipoprotein, optionally wherein the apolipoprotein is apolipoprotein A-I, apolipoprotein A-II, or apolipoprotein E. In some embodiments, the synthetic nanoparticle further comprises a cholesterol.

In some embodiments, the shell comprises a lipid monolayer or a lipid bilayer. In some embodiments, at least a portion of the lipid bilayer is covalently bound to the nanoparticle core.

In some embodiments, the nanoparticle core: (i) has a largest cross-sectional dimension of less than or equal to about 500 nanometers (nm), less than or equal to about 250 nanometers (nm), less than or equal to about 100 nanometers (nm), less than or equal to about 75 nanometers (nm), less than or equal to about 50 nanometers (nm), less than or equal to about 30 nanometers (nm), less than or equal to about 15 nanometers (nm), less than or equal to about 10 nanometers (nm), less than or equal to about 5 nanometers (nm), less than or equal to about 3 nanometers (nm); or (ii) has a diameter of about 5-30 nm, 5-20 nm, 5-15 nm, 5-10 nm, 8-13 nm, 8-12 nm, or 10 nm.

In some embodiments, the nanoparticle core has an aspect ratio of greater than about 1:1, 3:1, or 5:1.

In some embodiments, the lipids of the shell are phospholipids. In some embodiments, the phospholipids comprise 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoethanolamine (16:0 PE), 1,2-distearoyl-sn-glycero-3-phosphoethanolamine (18:0 PE), sphingomyelin, 1,2-dioleoyl-3-trimethylammonium-propane (DOTAP), or a combination thereof.

In some embodiments, the core is an organic core.

In some embodiments, the nanoparticle further comprises a DNA molecule. In some embodiments, the organic core comprises a hydrophobic phospholipid conjugated scaffold, optionally wherein the hydrophobic phospholipid conjugated scaffold is PL4. In some embodiments, the organic core comprises an amphiphilic DNA-linked small molecule-phospholipid conjugate (DNA-PL4).

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 provides a schematic of the synthesis of synthetic high-density lipoprotein nanoparticles (HDL NPs) using inorganic core scaffold. HDL NPs are made using a 5 nm diameter citrate stabilized gold nanoparticle scaffold surface-functionalized with apoA-I and a phospholipid layer.

FIGS. 2A-2B provide images showing the therapeutic effect of HDL NPs on wound healing of mouse corneal epithelium. Corneal images (FIG. 2A) and epithelial corneal wound closure percentage (FIG. 2B) in DIG mouse corneas treated with HDL NPs or control. Green fluorescence represents corneal wound. N=8. *p<0.05.

FIGS. 3A-3D demonstrate that HDL NP treatment reduces inflammation after alkali burn. Mice were treated with HDL NPs, control NPs, or PBS (topically) following 30 seconds of alkali burns. (FIG. 3A) Representative images. (FIG. 3B). Degree of haze. (FIGS. 3C-3D). H&E 7 days post burn. N=8.

FIG. 4 demonstrate that HDL NPs have anti-inflammation activity. Filter paper (1 mm) soaked in NaOH (1 m) was placed on the corneal surface of 6 week old WT mice for 30 seconds and then washed extensively with PBS. Corneas were topically treated with HDL NPs, control NPs (inert AuNP core, passivated with polyethyleneglycol (PEG)), or PBS daily for 7 d. Whole corneal tissues were dissected and total RNAs were isolated for RT-qPCR for inflammation-related genes at post injury day 1, 3, and 7 (N=8). *p<0.05. Unpaired t-tests were conducted.

FIG. 5 provides schematics of bioengineered synthetic organic core HDL NPs (ocHDL NPs) using organic (PL4 and DNA-PL4) core scaffold. Organic tetrahedral phospholipid (PL4) or PL4 with bioprogrammable DNA “arms” (DNA-PL4) are used as scaffolds for ocHDL NPs. Synthesis of ocHDL NPs made using organic scaffolds proceeds similar to the ones made using AuNPs.

FIG. 6 provides schematic of HDL NP eye drops.

FIG. 7A provides a graph of a standard curve of Vitamin D3 (calcitriol).

FIG. 7B-7C provide graphs showing the loading of Vitamin D3 (calcitriol) into oc-HDL-NPs at varying ratios of PL4 core to calcitriol (1:300, 1:100, and 1:50 of PL4:calcitrol) and Au core HDL NPs to calcitriol (1:100).

FIGS. 8A-8D provide graphs showing that topical administration of HDL-NPs loaded with Vitamin D3 reduces corneal inflammation after chemical injury (nitrogen mustard). Markers of inflammation were assessed by RT-PCR (FIGS. 8A-8C), and clinical scores were obtained (FIG. 8D).

FIG. 9 provides a graph showing that the HDL-NPs of the disclosure that are loaded with calcitriol are capable of delivering calcitriol to human corneal epithelial cells (HCECs) at elevated rates relative to calcitriol alone.

FIGS. 10A-10C provide graphs showing that topical administration of HDL-NPs loaded with Vitamin D3 reduces inflammation of the skin after chemical injury (nitrogen mustard). Percent change in skin thickness following treatment (FIGS. 10A-10B) and markers of inflammation as assessed by RT-PCR (FIG. 10C) demonstrate that HDL-NPs of the disclosure that are loaded with calcitriol are capable of reducing inflammation.

### DETAILED DESCRIPTION OF THE INVENTION

The present invention relates to high-density lipid nanoparticles (HDL NPs) (also referred to as synthetic nanoparticles) and/or HDL NPs comprising anti-inflammatory molecules (e.g., Vitamin D3) that are useful for reducing inflammation in the eye or skin of a subject (e.g., a subject having a disease or disorder that causes inflammation or is otherwise associated with inflammation of the eye or skin).

Vitamin D3 is a fat-soluble vitamin which promote bone health and maintains calcium homeostasis. In addition, vitamin D3 plays important roles in the eye and skin, where it has been shown to slow inflammation and the mitigate the dermatological effects of aging. Hence, efficient topical delivery of vitamin D3 to eye and skin cells could prevent or slow the progression of corneal disease and age-related skin phenotypes including thinning of the skin and dysregulated inflammation. Lipoprotein-mimetic nanoparticles (e.g., synthetic nanoparticles as described herein) exhibit potent anti-inflammatory properties in both the eye and skin. Accordingly, in some embodiments, the nanoparticles can be used as monotherapy for treatment of inflammation (e.g. to reduce inflammation) in the eye or skin; 2) as a delivery agent to deliver vitamin D3 to the eye or skin; or 3) co-administered with vitamin D3 for synergistic, anti-inflammatory effects in the eye and skin.

Moreover, lipoprotein-mimetic nanoparticles (e.g., synthetic nanoparticles as described herein) also exert anti-inflammatory and pro-regenerative effects on both the eye and skin. For instance, high-density lipoprotein mimics accelerate corneal re-epithelialization and wound-healing. Nanoparticles as described herein are also effective in treating inflammation (e.g., alkali burn-induced corneal inflammation).

Further, HDL-NPs as described herein exhibit valuable therapeutic properties such as: (i) endowment with a surface chemistry that closely mimics the physical and chemical features of natural HDLs; (ii) presence of apolipoproteins such as apoA-I; (iii) capability to bind with high-affinity to SR-B1; (iv) lack of toxicity to healthy cells in vitro or in vivo; (v) capable of being chemically engineered to display nearly any surface or core composition desired. The NPs described herein are also (i) highly stable; (ii) produce minimal adverse side effects; (iii) have tissue regenerative capabilities; and (iv) anti-inflammatory properties. These features endow HDL NPs with great translational potential and exemplify the impact that nanoparticles can have in medicine. Specifically, an HDL NP eye drop should be effective in treating dry eye or tear gland insufficiency, whose underlying etiology is inflammation. Since destruction of the stem cell niche due to persistent inflammation is thought to be an underlying cause of limbal stem cell deficiency, an HDL NP eye drop should be helpful in modulating and/or avoiding limbal stem cell deficiency (LSCD). Nitrogen and sulfur mustards (NM and SM) are devastating compounds that have been used as chemical warfare agents. Recently, it has been shown that Vitamin D3 treatment protected against SM toxicity and prevented SM-induced mortality. The eye is the most susceptible part of the body to the effects of mustard exposure, particularly SM. Following exposure, severe and extensive inflammation is triggered, which also can result in phenotypes mimicking LSCD. In some embodiments, delivery of HDL NPs and/or HDL NPs comprising anti-inflammatory molecules (e.g., Vitamin D3) to the eye is effective in treating corneal mustard keratopathy (CMK). In some embodiments, delivery of HDL NPs and/or HDL NPs comprising anti-inflammatory molecules (e.g., Vitamin D3) to the eye is effective in treating corneal mustard keratopathy (CMK) without the side-effects associated with steroidal treatments. Corneal keratopathy occurs in more than 70% of diabetic patients, which manifested in part, as persistent epithelial defects, and recurrent erosions. Therefore, HDL NPs and/or HDL NPs comprising anti-inflammatory molecules (e.g., Vitamin D3) can be effective prophylactically in patients with diabetes. Finally, HDL NPs and/or HDL NPs comprising anti-inflammatory molecules (e.g., Vitamin D3) are effective in treating a wide variety of ocular surface diseases such as chemical and thermal injury, long-term contact lens wear, severe chronic rosacea, Stevens-Johnson syndrome, atopic keratoconjunctivitis, bacterial keratitis and graft versus host disease.

As used herein, the terms “HDL-like”, “HDL-mimetic”, and “HDL mimic” are used interchangeably to refer to a synthetic HDL-NP of the disclosure. HDL-NPs are, in some embodiments, mimetics of high density lipoprotein particles. Thus, in some embodiments, HDL-NPs that are mimetics of high density lipoprotein particles do not require the presence of high density lipoproteins.

In some embodiments, the HDL NPs of the present invention bind to the receptor for mature HDLs, scavenger receptor type B1 (SR-B1), also referred to as SCARB1, which are used interchangeably herein (a high-affinity receptor for cholesterol-rich high-density lipoproteins (HDL)), and starve the malignant cells of cholesterol by preventing internalization of cholesteryl esters from natural HDLs and effluxing free cholesterol from the cell.

### Synthetic Nanoparticles

In some embodiments of the present disclosure, inflammation of the eye or skin of a subject is reduced by administering (e.g., topically administering) a synthetic nanoparticle as described herein. In some embodiments, the synthetic nanoparticle comprises a nanoparticle core, a shell, the shell comprising a lipid layer surrounding and attached to the nanoparticle core. In some embodiments, the synthetic nanoparticle further comprises a protein associated with the shell. Examples of synthetic nanoparticles useful for the present purposes are described below.

Examples of synthetic nanoparticles that can be used in the methods are described herein. The structure (e.g., synthetic nanoparticle, HDL NP) has a core and a shell surrounding the core. In embodiments in which the core is a nanoparticle, the core includes a surface to which one or more components can be optionally attached. For instance, in some cases, a core is a nanoparticle surrounded by shell, which shell includes an inner surface and an outer surface. The shell may be formed, at least in part, of one or more components, such as a plurality of lipids, which may optionally associate with one another and/or with surface of the core. For example, components may be associated with the core by being covalently attached to the core, physiosorbed, chemisorbed, or attached to the core through ionic interactions, hydrophobic and/or hydrophilic interactions, electrostatic interactions, van der Waals interactions, or combinations thereof. In one particular embodiment, the core includes a gold nanoparticle, and the shell is attached to the core through a gold-thiol bond.

In some embodiments, the HDL NPs of the present disclosure mimic HDL species using lipid-conjugated organic core scaffolds. The core design motif constrains and orients phospholipid geometry to facilitate the assembly of soft-core nanoparticles that are, in some embodiments, approximately 10 nm in diameter and resemble human HDLs in their size, shape, surface chemistry, composition and protein secondary structure. The HDL-like nanoparticles mimic the structure of native HDL with respect to size (˜10 nm), surface chemistry (˜20 mV zeta potential), and HDL protein secondary structure as determined by circular dichroism. Synthetic HDL-NPs have demonstrated promise as therapy for cardiovascular disease and cancer, among other indications.

In some embodiments, the synthesis of HDL mimetic nanoparticles using lipid-conjugated core scaffolds (HDL NPs) is accomplished in a two-step process: first, the core scaffolds are synthesized and purified; second, the particle is fabricated via supramolecular assembly of the core scaffold, free phospholipids, and the HDL-defining protein, apolipoprotein A1 (apo-A1). A variety of lipid-conjugated organic cores can theoretically be used for particle assembly. Herein, successful particle fabrication using a tetrahedral small molecule-phospholipid hybrid, called PL4 is used.

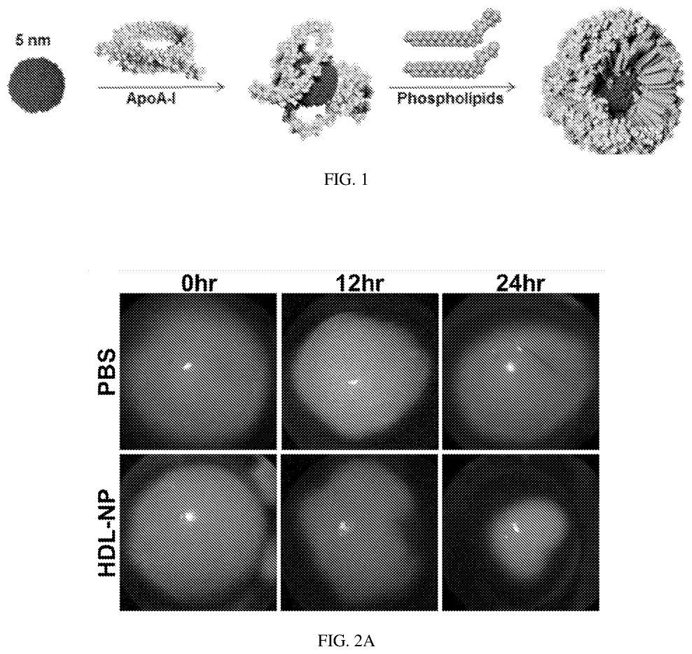

In some embodiments, an organic scaffold using a highly hydrophobic small molecule-phospholipid conjugate (PL4) was synthesized using copper-free click chemistry. Specifically, a headgroup-modified phospholipid harboring a ring-strained alkyne, 1,2-dipalmitoyl-sn-glycero-3-phosphoethan-olamine-N-dibenzocyclooctyl, was click coupled to tetrakis(4-az-idophenyl)methane, a small molecule with four terminal azides (SM-Az4) (FIGS. 1B-1C). As used herein, the terms “HDL-like”, “HDL-mimetic”, and “HDL mimic” are used interchangeably to refer to a synthetic HDL-NP of the disclosure.

In some aspects, the disclosure relates to a high-density lipoprotein nanoparticle (HDL-NP) comprising: (a) an organic core (core); (b) a shell surrounding and attached to the core wherein the core comprises a hydrophobic phospholipid conjugated scaffold (PL4); and (c) an anti-inflammatory molecule associated with one or more of the organic core or shell.

In some embodiments, the HDL-NP further comprises an apolipoprotein. In some embodiments, the apolipoprotein is apolipoprotein A-I, apolipoprotein A-II, or apolipoprotein E. In some embodiments, the apolipoprotein is apolipoprotein A-I (Apo-I).

The shell may be formed, at least in part, of one or more components, such as a plurality of lipids, which may optionally associate with one another and/or with surface of the organic core. For example, components (e.g., shell, lipid shell) may be associated with the organic core by being covalently or non-covalently attached to the organic core, physiosorbed, chemisorbed, or attached to the organic core through ionic interactions, hydrophobic and/or hydrophilic interactions, electrostatic interactions, van der Waals interactions, or combinations thereof. In some embodiments, the shell is non-covalently attached to the organic core. In some embodiments, the shell is attached to the organic core by hydrophobic interactions.

In some embodiments, an anti-inflammatory molecule is associated (e.g., by any of the means described herein) with the organic core. In some embodiments, the anti-inflammatory molecule is associated (e.g., by any of the means described herein) with the shell. In some embodiments, the anti-inflammatory molecule is associated (e.g., by any of the means described herein) to the organic core and the shell. In some embodiments, as described elsewhere herein, the HDL-NP comprises an apolipoprotein, in some such embodiments, the anti-inflammatory molecule is associated with an apolipoprotein. In some embodiments, the anti-inflammatory molecule is associated to the organic core and an apolipoprotein. In some embodiments, the anti-inflammatory molecule is associated to the organic a shell and an apolipoprotein. In some embodiments, the anti-inflammatory molecule is associated to an organic core, shell, and an apolipoprotein. In some embodiments, as described elsewhere herein, the HDL-NP comprises additional components, in some such embodiments, the anti-inflammatory molecule is associated with any additional component. In some embodiments, the anti-inflammatory molecule is associated to the outer layer of a shell. In some embodiments, the anti-inflammatory molecule is associated to the inner layer of a shell. In some embodiments, the attachment is by hydrophobic interactions. In some embodiments, the attachment is a non-covalent attachment.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E, and K), glycerides (e.g., monoglycerides, diglycerides, triglycerides) can be used to form portions of a structure described herein.

A portion of a structure described herein such as a shell or a surface of a nanoparticle may optionally include one or more alkyl groups, e.g., an alkane-, alkene-, or alkyne-containing species, that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6, or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclohexyl, and the like.

In some embodiments, the HDL-NP of the instant disclosure further comprises apolipoprotein. The apolipoprotein can be apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Additionally, or alternatively, a structure described herein may include one or more peptide analogues of an apolipoprotein, such as one described above. Of course, other proteins (e.g., non-apolipoproteins) can also be included in the nanoparticles described herein. In some embodiments, the apolipoprotein is apolipoprotein A-I.

The HDL-NP in some embodiments has an organic core scaffold. An organic core scaffold as used herein refers to non-metallic material, soft-core, having a 3-dimensional structure and charge sufficient to organize and hold a lipid layer in a stable shape. In some embodiments, the shape is spherical. A “spherical” shape or structure herein refers to a structure having a round or sphere-like structure. The structure does not need to be perfectly round or an exact sphere, but rather is an approximate sphere shape.

In some embodiments, the organic core scaffold comprises a hydrophobic small molecule-phospholipid conjugate (PL4). The hydrophobic small molecule-phospholipid conjugate comprises any small molecule capable of being linked to a phospholipid. In some embodiments, the small molecule is tetrakis(4-az-idophenyl)methane.

In some embodiments, the phospholipid may be a headgroup-modified phospholipid. In some embodiments, the headgroup-modified phospholipid comprises a ring-strained alkyne, 1,2-dipalmitoyl-sn-glycero-3-phosphoethan-olamine-N-dibenzocyclooctyl.

In other embodiments, the organic core scaffold comprises an amphiphilic DNA-linked small molecule-phospholipid conjugate (DNA-PL4). The DNA (or any other nucleic acid, including modified and naturally occurring nucleic acids) provides a unique link between the phospholipid and small molecule. It is advantageous to use DNA because the size of the DNA and thus the core may be easily controlled by altering the length of the DNA strand. In some embodiments the DNA is 5-50 nucleotides in length In other embodiments the DNA is 5-45, 5-40, 5-35, 5-30, 5-25, 5-20, 5-17, 5-16, 5-15, 5-14, 5-13, 5-12, 5-11, 5-10, 5-9, 5-8, 5-7, 6-45, 6-40, 6-35, 6-30, 6-25, 6-20, 6-17, 6-16, 6-15, 6-14, 6-13, 6-12, 6-11, 6-10, 6-9, 6-8, 6-7, 7-45, 7-40, 7-35, 7-30, 7-25, 7-20, 7-17, 7-16, 715, 7-14, 7-13, 7-12, 7-11, 7-10, 7-9, 7-8, 8-45, 8-40, 8-35, 8-30, 8-25, 8-20, 8-17, 8-16, 8-15, 8-14, 8-13, 8-12, 8-11, 8-10, 8-9, 9-45, 9-40, 9-35, 9-30, 9-25, 9-20, 9-17, 9-16, 9-15, 9-14, 9-13, 9-12, 9-11, or 9-10 nucleotides in length.

In some embodiments, the DNA is a double stranded oligonucleotide. In some embodiments, the DNA is a double stranded oligonucleotide of 8-15 nucleotides in length. In some embodiments, the DNA is a double stranded oligonucleotide of 9 nucleotides in length.

In some embodiments, a first single strand of the double stranded DNA is linked to a phospholipid and forms a ssDNA-phospholipid conjugate (ssDNA-PL). In some embodiments, a second strand of the double stranded DNA, complementary to the first strand of the double stranded DNA is linked to a small molecule. In some embodiments, the small molecule is a tetrahedral small molecule and the small molecule linked to the DNA forms a tetrahedral small molecule-DNA hybrid (SMDH4). In some embodiments, the SMDH4 is linked to the ssDNA-PL through hydrogen bonding between the complementary single strands of DNA.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, shell includes a lipid bilayer that is arranged to sequester cholesterol and/or control cholesterol efflux out of cells, as described herein.

It should be understood that a shell that surrounds a core need not completely surround the core, although such embodiments may be possible. For example, the shell may surround at least 50%, at least 60%, at least 70%, at least 80%, at least 90%, or at least 99% of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. As described herein, the shell may include one layer of material, or multilayers of materials in some embodiments.

In certain embodiments that synthetic nanoparticle may further include one or more agents, such as an anti-inflammatory molecule (e.g., Vitamin D3). In some embodiments, an anti-inflammatory molecule is Vitamin D3, a Vitamin D derivative or related compound, a Vitamin D precursor, or calcitriol. In some embodiments, an anti-inflammatory molecule is any small molecule. In some embodiments, an anti-inflammatory molecule is a protein. In some embodiments, an anti-inflammatory molecule is nucleic acid. In some embodiments, an anti-inflammatory molecule is a nonsteroidal anti-inflammatory drugs (NSAIDs) (e.g., aspirin, ibuprofen, naproxen), antileukotrines (e.g., arachidonate 5-lipoxygenase, cysteinyl leukotriene receptors), or an immune selective anti-inflammatory derivatives. The agent may be a diagnostic agent (which may also be known as an imaging agent), a therapeutic agent, or both a diagnostic agent and a therapeutic agent. In certain embodiments the diagnostic agent is a tracer lipid. Tracer lipids may comprise a chromophore, a biotin subunit, or both a chromophore and a biotin subunit. The synthetic nanoparticles (e.g. HDL NPs) can also be functionalized with other types of cargo such as nucleic acids. In certain embodiments the therapeutic agent may be a nucleic acid, antiviral agent, antineurological agent, or antirheumatologic agent.

The one or more agents may be associated with the core, the shell, or both; e.g., they may be associated with surface of the core, inner surface of the shell, outer surface of the shell, and/or embedded in the shell. For example, one or more agents may be associated with the core, the shell, or both through covalent bonds, physisorption, chemisorption, or attached through ionic interactions, hydrophobic and/or hydrophilic interactions, electrostatic interactions, van der Waals interactions, or combinations thereof.

In some cases, the synthetic nanoparticle is a synthetic cholesterol binding nanoparticle having a binding constant for cholesterol, Kd. In some embodiments, Kd is less than or equal to about 100 μM, less than or equal to about 10 μM, less than or equal to about 1 μM, less than or equal to about 0.1 μM, less than or equal to about 10 nM, less than or equal to about 7 nM, less than or equal to about 5 nM, less than or equal to about 2 nM, less than or equal to about 1 nM, less than or equal to about 0.1 nM, less than or equal to about 10 μM, less than or equal to about 1 μM, less than or equal to about 0.1 μM, less than or equal to about 10 fM, or less than or equal to about 1 fM. Methods for determining the amount of cholesterol sequestered and binding constants are known in the art.

The core of the nanoparticle may have any suitable shape and/or size. For instance, the core may be substantially spherical, non-spherical, oval, rod-shaped, pyramidal, cube-like, disk-shaped, wire-like, or irregularly shaped. In preferred embodiments of the present invention, the core is less than or equal to about 5 nm in diameter. The core (e.g., a nanoparticle core or a hollow core) may have a largest cross-sectional dimension (or, sometimes, a smallest cross-section dimension, or diameter) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, less than or equal to about 10 nm, less than or equal to about 5 nm, less than or equal to about 4 nm, less than or equal to about 3 nm, less than or equal to about 2 nm, or less than or equal to about 1 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

In embodiments in which core includes a nanoparticle core, the nanoparticle core may be formed from any suitable material. In preferred embodiments, the core is formed from gold (e.g. made of gold (Au)). In some embodiments, the core is formed of a synthetic material (e.g., a material that is not naturally occurring, or naturally present in the body). In one embodiment, a nanoparticle core comprises or is formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 wt %, 5 wt %, 10 wt %, 25 wt %, 50 wt %, 75 wt %, 90 wt %, or 99 wt %. In one embodiment, the core is formed of 100 wt % inorganic material. The nanoparticle core may, in some cases, be in the form of a quantum dot, a carbon nanotube, a carbon nanowire, or a carbon nanorod. In some cases, the nanoparticle core comprises, or is formed of, a material that is not of biological origin. In some embodiments, a nanoparticle includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

Those of ordinary skill in the art are familiar with techniques to determine sizes of structures and particles. Examples of suitable techniques include dynamic light scattering (DLS) (e.g., using a Malvern Zetasizer instrument), transmission electron microscopy, scanning electron microscopy, electroresistance counting and laser diffraction. Other suitable techniques are known to those of ordinary skill in the art. Although many methods for determining sizes of nanoparticles are known, the sizes described herein (e.g., largest or smallest cross-sectional dimensions, thicknesses) refer to ones measured by dynamic light scattering.

The shell of a structure described herein may comprise any suitable material, such as a hydrophobic material, a hydrophilic material, and/or an amphiphilic material. Although the shell may include one or more inorganic materials such as those listed above for the nanoparticle core, in many embodiments the shell includes an organic material such as a lipid or certain polymers. The components of the shell may be chosen, in some embodiments, to facilitate the sequestering of cholesterol or other molecules. For instance, cholesterol (or other sequestered molecules) may bind or otherwise associate with a surface of the shell, or the shell may include components that allow the cholesterol to be internalized by the structure. Cholesterol (or other sequestered molecules) may also be embedded in a shell, within a layer or between two layers forming the shell.

The components of a shell may be charged, e.g., to impart a charge on the surface of the structure, or uncharged. In some embodiments, the surface of the shell may have a zeta potential of greater than or equal to about −75 mV, greater than or equal to about −60 mV, greater than or equal to about −50 mV, greater than or equal to about −40 mV, greater than or equal to about −30 mV, greater than or equal to about −20 mV, greater than or equal to about −10 mV, greater than or equal to about 0 mV, greater than or equal to about 10 mV, greater than or equal to about 20 mV, greater than or equal to about 30 mV, greater than or equal to about 40 mV, greater than or equal to about 50 mV, greater than or equal to about 60 mV, or greater than or equal to about 75 mV. The surface of the shell may have a zeta potential of less than or equal to about 75 mV, less than or equal to about 60 mV, less than or equal to about 50 mV, less than or equal to about 40 mV, less than or equal to about 30 mV, less than or equal to about 20 mV, less than or equal to about 10 mV, less than or equal to about 0 mV, less than or equal to about −10 mV, less than or equal to about −20 mV, less than or equal to about −30 mV, less than or equal to about −40 mV, less than or equal to about −50 mV, less than or equal to about −60 mV, or less than or equal to about −75 mV. Other ranges are also possible. Combinations of the above-referenced ranges are also possible (e.g., greater than or equal to about −60 mV and less than or equal to about −20 mV). As described herein, the surface charge of the shell may be tailored by varying the surface chemistry and components of the shell.

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanoparticle may optionally include one or more alkyl groups, e.g., an alkane-, alkene-, or alkyne-containing species that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclohexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanoparticle core directly or via a linker. For example, where inert metals are used to form a nanoparticle core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, styrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanoparticle core and/or with each other. For instance, the amphiphilic material may include a hydrophilic group that points towards the core and a hydrophobic group that extends away from the core, or the amphiphilic material may include a hydrophobic group that points towards the core and a hydrophilic group that extends away from the core. Bilayers of each configuration can also be formed.

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and may be useful to include in structures described herein. Additionally, or alternatively, a structure described herein may include one or more peptide analogues of an apolipoprotein, such as one described above. A structure may include any suitable number of, e.g., at least 1, 2, 3, 4, 5, 6, or 10, apolipoproteins or analogues thereof. In certain embodiments, a structure includes 1-6 apolipoproteins, similar to a naturally occurring HDL particle. Of course, other proteins (e.g., non-apolipoproteins) can also be included in structures described herein.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell. Furthermore, such components can be used, in some embodiments, to facilitate the sequestration, exchange and/or transport of materials (e.g., proteins, peptides, polypeptides, nucleic acids, nutrients) from one or more components of a subject (e.g., cells, tissues, organs, particles, fluids (e.g., blood), and portions thereof) to a structure described herein, and/or from the structure to the one or more components of the subject. In some cases, the components have chemical and/or physical properties that allow favorable interaction (e.g., binding, adsorption, transport) with the one or more materials from the subject.

### Combinations

In some embodiments the HDL NP disclosed herein is co-formulated with or administered in conjunction with an anti-inflammatory molecule such as Vitamin D3. In some embodiments, an anti-inflammatory molecule is Vitamin D3, a Vitamin D derivative or related compound, a Vitamin D precursor, or calcitriol. In some embodiments, an anti-inflammatory molecule is any small molecule. In some embodiments, an anti-inflammatory molecule is a protein. In some embodiments, an anti-inflammatory molecule is nucleic acid. In some embodiments, an anti-inflammatory molecule is a nonsteroidal anti-inflammatory drugs (NSAIDs) (e.g., aspirin, ibuprofen, naproxen), antileukotrines (e.g., arachidonate 5-lipoxygenase, cysteinyl leukotriene receptors), or an immune selective anti-inflammatory derivatives.

The HDL NP is administered in conjunction with an anti-inflammatory molecule in any manner in which the compounds are both delivered to a subject. For instance, an HDL NP and an anti-inflammatory molecule may be co-administered together at the same time or at different times. The two compounds may be administered at the same site or different sites, using the same route of administration or different routes of administrations. In some embodiments the HDL NP may be administered before the anti-inflammatory molecule, such as for instance about 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 1, 2, 3, 4, 5, 6, or 7 days, 1, 2, 3, 4, 5, 6, 7, 8, 9, or 10 weeks or 1 month, 3 months or 6 months. In other embodiments the HDL NP may be administered after the anti-inflammatory molecule, such as for instance about 5 minutes, 10 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 1, 2, 3, 4, 5, 6, or 7 days, 1, 2, 3, 4, 5, 6, 7, 8, 9, or 10 weeks or 1 month, 3 months or 6 months. The HDL NP and anti-inflammatory molecule may be administered multiple times in various cycles of administration.

### Pharmaceutical Compositions

As described herein, the synthetic nanoparticles may be used in “pharmaceutical compositions” or “pharmaceutically acceptable” compositions (also referred to as drugs), which comprise a therapeutically effective amount of one or more of the structures described herein, formulated together with one or more pharmaceutically acceptable carriers, additives, and/or diluents. The pharmaceutical compositions described herein may be useful for treating diseases or disorders associated with inflammation. It should be understood that any suitable structures described herein can be used in such pharmaceutical compositions, including those described in connection with the figures. In some cases, the structures in a pharmaceutical composition have a nanoparticle core comprising an inorganic material and a shell substantially surrounding and attached to the nanoparticle core.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, and sublingual, boluses, powders, granules, pastes for application to the tongue; as a sterile solution or suspension, or sustained-release formulation; spray applied to the oral cavity; for example, as cream or foam. In some embodiments, the pharmaceutical compositions are formulated for topical delivery to the eye. In some embodiments, the pharmaceutical compositions are formulated for topical delivery to the skin. In some embodiments, the pharmaceutical compositions are formulated for intradermal or transdermal delivery to the skin. In some embodiments, the pharmaceutical compositions are formulated for intraocular delivery to the eye.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Pharmaceutical compositions described herein include those suitable for oral administration. The formulations may conveniently be presented in unit dosage form and may be prepared by any methods well known in the art of pharmacy. The amount of active ingredient that can be combined with a carrier material to produce a single dosage form will vary depending upon the host being treated, and the particular mode of administration. The amount of active ingredient that can be combined with a carrier material to produce a single dosage form will generally be that amount of the compound that produces a therapeutic effect. Generally, this amount will range from about 1% to about 99% of active ingredient, from about 5% to about 70%, or from about 10% to about 30%.

The inventive compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient. An inventive structure may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein (e.g., for rectal or vaginal administration) may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the body and release the structures.

The active compound may be mixed under sterile conditions with a pharmaceutically-acceptable carrier, and with any preservatives, buffers, or propellants, which may be required.

The pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

### Therapeutically Effective Amount

The phrase “therapeutically effective amount” as used herein means that amount of a material or composition comprising an inventive structure that is effective for producing some desired therapeutic effect in a subject at a reasonable benefit/risk ratio applicable to any medical treatment. Accordingly, a therapeutically effective amount may, for example, prevent, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

### Subject

As used herein, a “subject” or a “patient” refers to any mammal (e.g., a human), for example, a mammal that may be susceptible to a disease or bodily condition such as the secondary diseases or conditions disclosed herein. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition. In some embodiments, a subject may be diagnosed with, or otherwise known to have, a disease or bodily condition associated with abnormal lipid levels, as described herein. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the invention may be administered to diagnose or prevent further development of the disease or bodily condition.

In some embodiments, the subject has or is suspected of having an inflammatory disease or disorder. In some embodiments, the inflammatory disease or disorder is selected from the group consisting of: corneal inflammation, corneal regeneration, ocular inflammation, age-related skin deterioration, psoriasis, atopic dermatitis, and ocular surface diseases. In some embodiments, the ocular surface diseases are selected from the group consisting of: chemical and thermal injury, long-term contact lens wear, severe chronic rosacea, Stevens-Johnson syndrome, atopic keratoconjunctivitis, bacterial keratitis and graft versus host disease.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Methods

In some embodiments, the eye or skin of a subject is administered any of the compositions described herein (e.g., synthetic nanoparticles). The compositions disclosed herein may be administered by any administration route known in the art. For example, in some embodiments, one of ordinary skill in the art, may administer a composition via conventional routes, e.g., administered orally, parenterally, by inhalation spray, topically, transdermally, intraocularly, rectally, nasally, buccally, vaginally or via an implanted reservoir.

In some embodiments, the eye or skin of a subject is administered a composition (e.g., synthetic nanoparticle) at least once. In some embodiments, a subject receives multiple administrations, or a cell is contacted multiple times. For example, without limitation, the subject may receive at least 2 administrations (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more). In some embodiments, the administrations are irregularly spaced (e.g., not an equal duration of time between administrations). In some embodiments, the administrations are equally spaced (e.g., an equal duration of time between administrations). In some embodiments, the eye or skin of a subject receives at least one administration per month. In some embodiments, the eye or skin of a subject receives at least one administration per week. In some embodiments, the eye or skin of a subject receives at least one administration per day. In some embodiments, the eye or skin of a subject receives at least two administrations per day. In some embodiments, where there is more than one administration, the administrations are of the same route. In some embodiments, where there is more than one administration, the administrations are of different routes.

### EXAMPLES

### Example 1: HDL NPs Enter Cells and Tissues of the Cornea

SR-B1 is the major receptor that controls the selective uptake of HDL cargo into cells. Single cell RNA sequencing showed that SR-B1 mRNA was detected in all cell populations of the mouse cornea. Consistently, SR-B1 protein was observed on primary human corneal epithelial cells (HCECs) as well as human and mouse corneal and limbal epithelia. SR-B1 was also present on the stromal keratocytes. In a series of in vitro and in vivo experiments, HDL NPs were demonstrated to enter HCECs and intact mouse corneas. Specifically, following the addition of HDL NPs to HCECs, non-membrane-bound gold particles were readily observed, free in the cytoplasm; few if any particles were detected in the nucleus or in the proximity of the cell membrane. More importantly, twenty-four hours after a fluorescent HDL NP conjugate in PBS was topically applied to intact mouse corneas, fluorescent signal was readily detected in the superficial and basal corneal epithelial cells as well as in the stromal keratocytes.

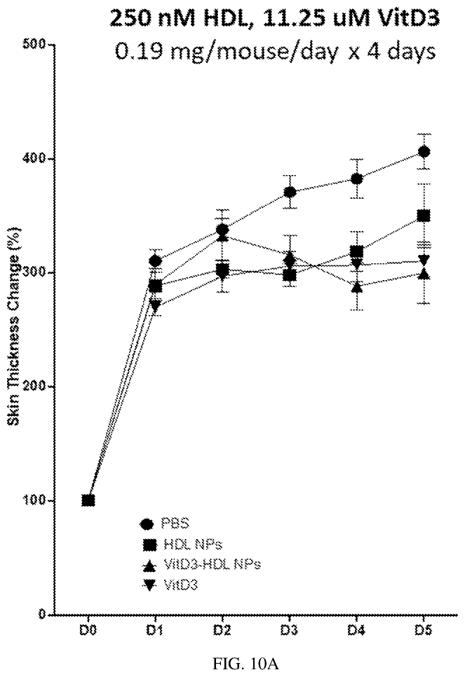

Furthermore, it was found that HDL NPs loaded with Vitamin D3 (Calcitriol) (“VitD-PL4-HDL-NP”) were capable of delivering high levels of Vitamin D3 to HCECs. HCECs were incubated with (i) VitD-PL4-HDL-NPs; (ii) empty HDL NP and Vitamin D3 (“PL4-HDL-NP+Calcitriol”); or (iii) Calcitriol for eight hours. The concentration of calcitriol uptake in the cells was measured at 30 min, 1 hour, 2 hours, 4 hours, and 8 hours after initial incubation. As shown in FIG. 9, HDL NPs loaded with Vitamin D3 delivered higher concentrations (>5000 ng/mL calcitriol after 8 hours of incubation) than the PL4-HDL-NP+Calcitriol or Calcitriol only experimental groups.

These data demonstrate that the HDL NPs of the disclosure provide high levels of delivery or administration of anti-inflammatory molecules (e.g., Vitamin D3) to target cells.

### Example 2: HDL NPs Accelerate Re-Epithelialization In Vivo

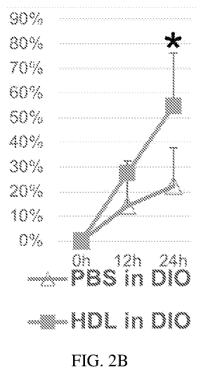

Using a diet-induced obesity (DIO) mouse model, which has an impaired wound healing response, HDL NPs applied topically to the corneal surface after a debridement wound sealed wounds significantly faster than controls (FIGS. 2A-2B). Such a positive effect on wound closure is due, in part, by HDL NPs up-regulation of the Akt signaling pathway, which is involved in re-epithelialization. Furthermore, Akt signaling regulates actin remodeling and cell migration. Thus, it is not surprising that a marked increase in F-actin was observed at the leading edge of the HDL NP-treated migrating cells compared with the control NP-treated cells. This suggests that HDL NPs are positive regulators of F-actin polymerization during the initial migratory phase of cell migration. Finally, phosphorylation of EphA2 at S897 via Akt can signal an increase in cell migration through the reorganization of actin filaments at the leading edge of a migrating sheet. Treatment of cells with HDL NPs resulted in a dramatic increase in p-EphA2-S897 expression compared with control NPs, providing compelling evidence that HDL NPs positively affect cell migration via targeting EphA2.

### Example 3: HDL NPs are Effective in Treating Alkali Burn-Induced Corneal Inflammation

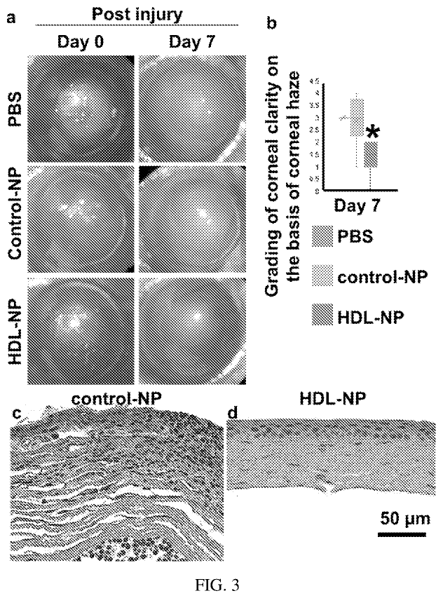

In the alkaline burn model to induce an inflammatory response in mouse corneas, the corneal epithelium, stromal, and inflammatory cells are involved in the injury, repair, and wound healing processes, which are accompanied by the production of numerous cytokines. Following such a wounding protocol, mice were treated daily for 4 days with a topical solution of HDL NPs, control NPs in PBS, or PBS. By day 7 post-wounding, PBS- and control NP-treated corneas remained opaque whereas the HDL NP-treated mice showed a 40-50% (p<0.05) improvement in corneal opacity and surface integrity (FIGS. 3A-3B). Control NP treated corneas displayed a range of thickened and disorganized corneal epithelia as well as a wide spectrum of stromal alterations, ranging from a stroma filled with inflammatory cells (FIG. 3C) to randomly oriented collagen bundles resulting in a disorganized appearance. In contrast the HDL NP-treated corneas displayed a well-organized stratified epithelium (FIG. 3D) and a stroma with collagen bundles highly organized in a plywood-like fashion that were relatively devoid of inflammatory cells. In some HDL NP treatment groups, stromal keratocytes were prominent.

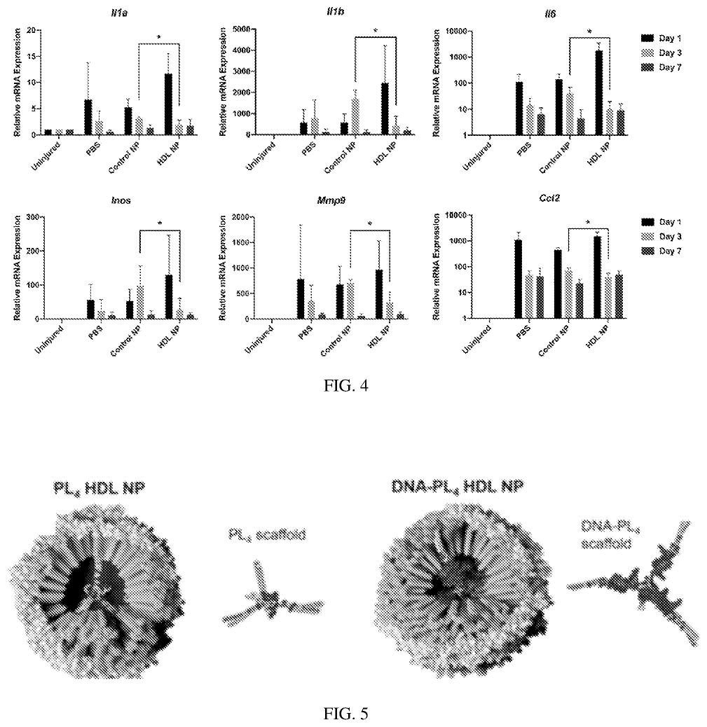

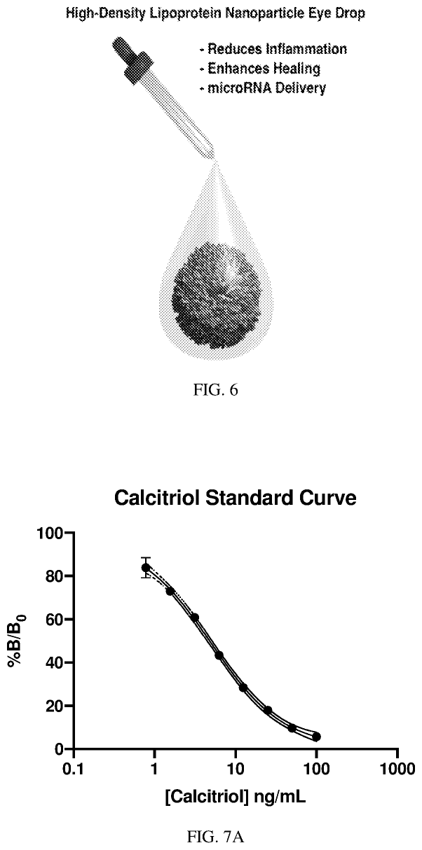

Immune cell recruitment after corneal injury is mediated by proinflammatory cytokines released from epithelial cells and keratocytes at the injured site. IL-1, IL-6 and TNF are important and aid in attracting neutrophils, which are the first cells infiltrating the cornea after injury. Shortly after neutrophils enter the cornea, macrophages extravasate from the limbal vessels, infiltrate the stroma from superficial to deeper layers and migrate towards the center of the cornea. Macrophages aid in corneal wound closure by secreting TGFβ to promote the differentiation of fibroblasts to myofibroblasts. In addition to removal of debris and apoptotic cells, macrophages are essential mediators of angiogenesis after severe and prolonged corneal injury. Several chemokines and their receptors have been identified in the inflamed cornea. CXCL1, CXCL8 and MCP-1/CCL2 mRNA levels were found to be elevated in human inflamed corneas. Additionally, CCR7 and its ligand CCL21 were upregulated in inflamed corneas, mediating MHC II+ cell recruitment. Evaluation of the alkali burn-induced cytokine expression pattern after treatment with HDL NPs revealed that day 1 post-injury, Il1a, Il1b, Il6 and Ccl2 were most highly expressed (FIG. 4), which is consistent with an initial stage of inflammation. By day 3, HDL NP treatment significantly reduced the expression levels of Il1a, Il1b, Il6, Inos, Mmp9 and Ccl2 when compared with control NPs (FIG. 4). Chemokines such as CCL2 play important roles in the recruitment of macrophages to the site of injury during an inflammatory event, as well as the inflammatory mediator iNOS, which is associated with activated macrophages. Elevated levels of Gelatinase or MMP-9 are associated with numerous diseases of the cornea and can facilitate corneal ulceration. All genes evaluated, returned to pre-treatment levels by day 7 (FIG. 4). Collectively, these findings strongly indicate that topical application of HDL NPs to the corneal surface following a chemical burn can aid in attenuating the inflammatory response.

### Example 4: Delivery of HDL NP and HDL NP Comprising Vitamin D3 to the Eye

Vitamin D3 is well-recognized as an immunomodulator through direct inhibition of NF-κB activation, suppression of TNF-α and iNOS expression, as well as activation of autophagy. Vitamin D3 is converted intracellularly to the active form in macrophages, a critical cell population activated following stress that exacerbates local cellular inflammation. The presence of the Vitamin D receptor (VDR) was detected in the human corneal epithelium, as well as the corneal endothelium. Additionally, the presence of vitamin D hydroxylases (CYP27B1, CYP27A1, CYP2R1, and CYP24A1) are present in corneal epithelial and endothelial cell lines, indicative that these cells have the ability to initiate and regulate Vitamin D3 metabolism. With respect to corneal inflammation, topical administration of Vitamin D3 to sutured mouse corneas (a model for inflammation) inhibited Langerhans cell migration and maturation, while delaying neovascularization in the central cornea. Vitamin D3 protected corneal graft rejection by inhibiting the proinflammatory cytokines IL-1α and TNF-α, in rats. In vitro studies in corneal epithelial cells demonstrated immunomodulatory activity of Vitamin D3 via attenuation of proinflammatory mediators while increasing antimicrobial peptides and anti-pseudomonas activity. These data show that Vitamin D3 is useful for treatment of a variety of corneal inflammatory diseases because it targets macrophages and suppresses inflammation without the side effects commonly associated with long-term steroid usage. In addition, Vitamin D3 can activate autophagy, a critical stress response process to differentiate macrophages towards an anti-inflammatory repair phenotype. Autophagy has an important role in maintaining limbal epithelial stem cell homeostasis.

Given the anti-inflammatory properties of HDL NPs and Vitamin D3, an eye drop for topical delivery to the eye containing HDL NP comprising Vitamin D3 eye drop is effective in reducing inflammation in the anterior segment. HDL NPs made with various cores and phospholipids can also be formulated with Vitamin D3.

### Example 5: HDL NPs Function to Reduce Inflammation in the Eye

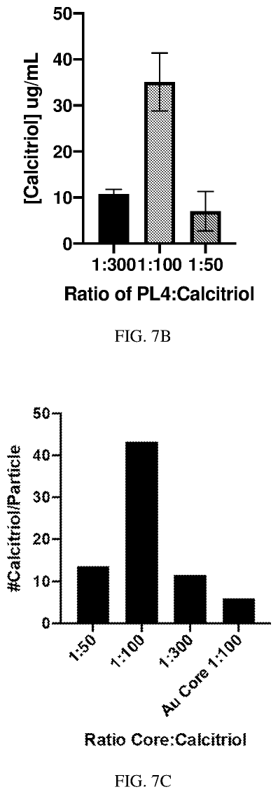

Vitamin D3 (calcitriol) was loaded into HDL NPs at varying ratios of PL4 core to calcitriol (1:300, 1:100, and 1:50 of PL4:calcitrol). An ELISA assay was used to quantify the amount, and verify presence of calcitriol after particle synthesis (FIGS. 7B-7C). The 1:100 PL4:calcitriol ratio resulted in maximum loading, yielding about 43 calcitriol molecules per nanoparticle. The use of a gold (Au) core in the nanoparticle provided about 6 calcitriol molecules per nanoparticle. A calcitriol standard curve was used for the quantification (FIG. 7A), where B₀ represents the maximum binding amount in the absence of free analyte and % B/B₀ represents the ratio of the absorbance of a particular sample or standard well to that of the maximum binding sample.

Size exclusion chromatography (SEC) was used to evaluate the hydrodynamic diameter of nanoparticle populations and their size distribution profiles. SEC demonstrated that a dominant population of nanoparticles with hydrodynamic diameter similar to native human HDLs was produced. SEC also revealed a secondary peak consistent with free, unbound Vitamin D3.

Mice were subjected to corneal injury by exposure to nitrogen mustard, and subsequently treated with either phosphate buffered saline (PBS) control, empty HDL-NP (i.e., without any additional anti-inflammatory molecule), Vitamin D3, or Vitamin D3-loaded HDL NPs.

A 1 mm diameter sterile #1 filter paper disc with either NM solution (2% NM in PBS+3% DMSO) or vehicle (PBS+3% DMSO) was placed onto the central corneas of mouse eyes (n=10) for 5 min. Clinical images of mouse eyes were taken daily. The initial degree of corneal epithelial wounding was assessed by topically applying 20 μL of 0.5% fluorescein in PBS and imaging the wound under cobalt blue illumination. Following this assessment, mice were treated topically, twice daily for 3 days with: (i) 5 μl of an HDL NP solution (1 μM in PBS); (ii) 5 μl of a Vitamin D3-loaded HDL NP; (iii) phosphate-buffered saline (PBS, control); or (iv) Vitamin D3. Prior to sacrifice, all mice were clinically evaluated daily for corneal clarity based on the degree of haze and surface integrity as determined by exclusion of fluorescein dye. Mice also received an intraperitoneal injection of BrdU (50 mg/kg) 1 hr prior to sacrifice. Mice (n=10) were sacrificed at 3 and 7 days post treatment, corneas were isolated and prepared for qPCR and histological examination.

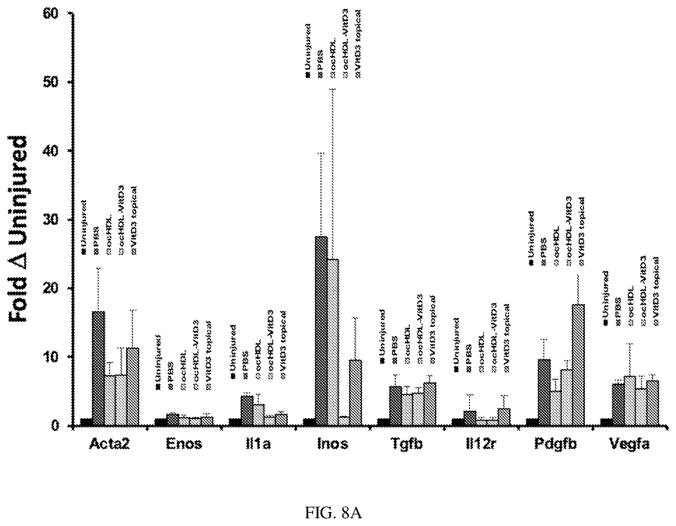

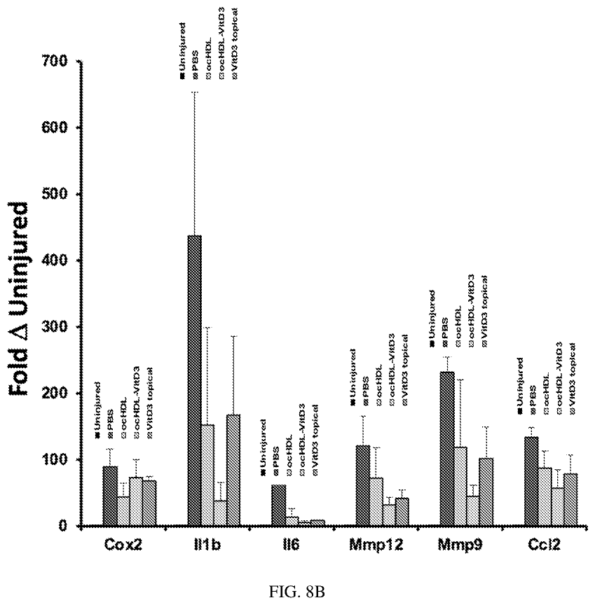

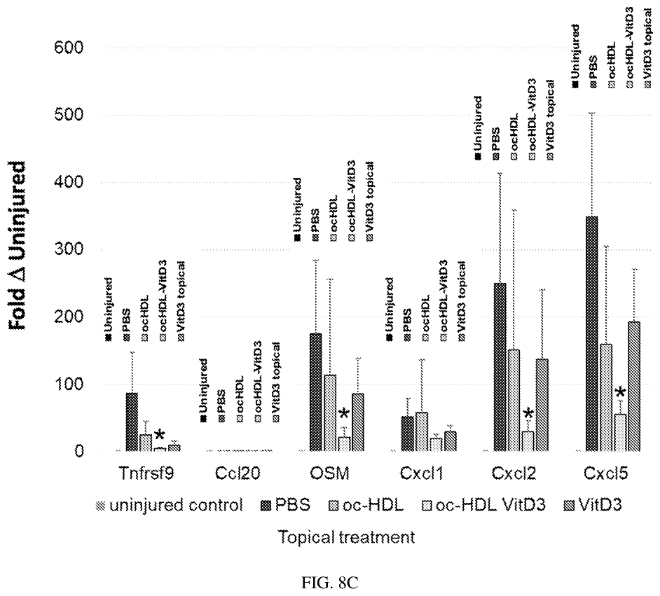

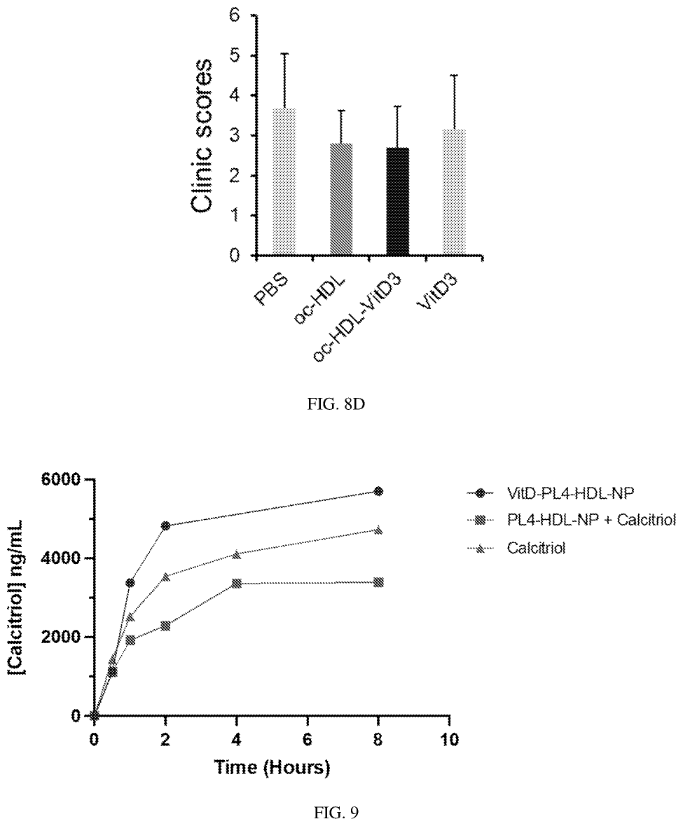

Markers of inflammation were assessed by RT-PCR (FIGS. 8A-8C), and clinical scores were obtained (FIG. 8D). Higher clinical scores indicate more severe injury phenotype. Vitamin D3-loaded HDL NPs consistently suppressed markers of inflammation (e.g., IL1a, IL1b, TGFb, PDGFb, IL6, MMP12, CCL2, iNOS, MMP9), often to a greater degree than either Vitamin D3 alone or HDL NPs alone, suggesting synergistic behavior. Moreover, HDL NPs and Vitamin D3-loaded HDL NPs produced the most favorable clinical scores.

### Example 5: HDL NPs Function to Reduce Inflammation of the Skin

Mice were subjected to skin injury by exposure to nitrogen mustard (chemical injury) and subsequently topically treated with either phosphate buffered saline (PBS) control, empty HDL-NP (i.e., without any additional anti-inflammatory molecule), Vitamin D3, or Vitamin D3-loaded HDL NPs

Mice were first shaved and depilated, and the shaved/depilated skin area was then exposed to a 1 mm diameter sterile #1 filter paper disc with a 2% nitrogen mustard solution in DMSO for 5 min to induce chemical injury (Day 0 of the experiment). Shortly thereafter, on Day 0 of the experiment, the shaved/depilated skin area was topically treated with an experimental treatment as described in Table 1. The topical treatment was repeated on Days 1, 2, and 3. The skin area was monitored on each of Days 0-5 to obtain skin thickness measurements, before the mice were sacrificed on Day 5. The average percent increase in skin thickness measurement on Day 5 (relative to Day 0) across all mice of a treatment group are also provided in Table 1.

TABLE 1
Treatment Groups

| | | Amount of | | Amount of | | | Percent |
|---|---|---|---|---|---|---|---|
| | | Calcitriol | | HDL-NP | | | Increase |
| | | applied | | applied | | | in Skin |
| | Molecular | per mouse | | per mouse | | Number | Thickness |
| Group | intervention | per day | | per day | | of mice | (Day 5) |
| 1 | Phosphate- | 0 | mg | 0 | nM | 6 | 398% |
| | buffered saline | | | | | | |
| | (PBS) | | | | | | |
| 2 | Calcitriol | 0.075 | mg | 0 | nM | 6 | 345% |
| 3 | Empty HDL-NP | 0 | mg | 100 | nM | 6 | 380% |
| 4 | HDL-NP loaded | 0.075 | mg | 100 | nM | 6 | 355% |
| | with calcitriol | | | | | | |
| 5 | Phosphate- | 0 | mg | 0 | nM | 6 | 353% |
| | buffered saline | | | | | | |
| | (PBS) | | | | | | |
| 6 | Calcitriol | 0.1 | mg | 0 | nM | 6 | 301% |
| 7 | Empty HDL-NP | 0 | mg | 150 | nM | 6 | 347% |
| 8 | HDL-NP loaded | 0.1 | mg | 150 | nM | 6 | 377% |
| | with calcitriol | | | | | | |
| 9 | Phosphate- | 0 | mg | 0 | nM | 8 | 406% |
| | buffered saline | | | | | | |
| | (PBS) | | | | | | |
| 10 | Calcitriol | 0.19 | mg | 0 | nM | 8 | 310% |
| 11 | Empty HDL-NP | 0 | mg | 250 | nM | 8 | 350% |
| 12 | HDL-NP loaded | 0.19 | mg | 250 | nM | 8 | 300% |
| | with calcitriol | | | | | | |
| 13 | Phosphate- | 0 | mg | 0 | nM | 6 | 402% |
| | buffered saline | | | | | | |
| | (PBS) | | | | | | |
| 14 | Calcitriol | 0.56 | mg | 0 | nM | 8 | 288% |
| 15 | Empty HDL-NP | 0 | mg | 750 | nM | 9 | 414% |
| 16 | HDL-NP loaded | 0.56 | mg | 750 | nM | 8 | 273% |
| | with calcitriol | | | | | | |

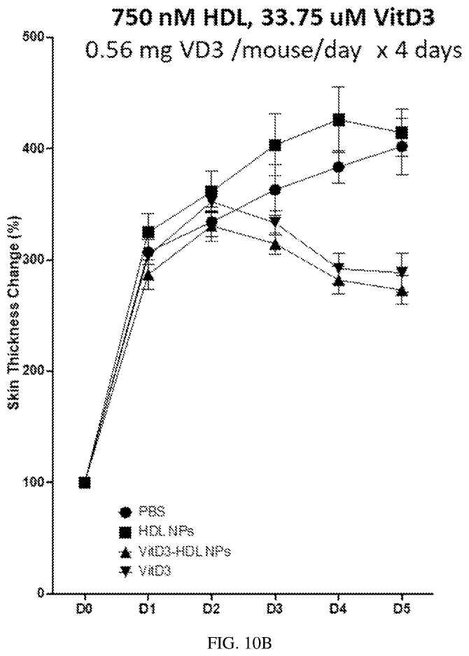

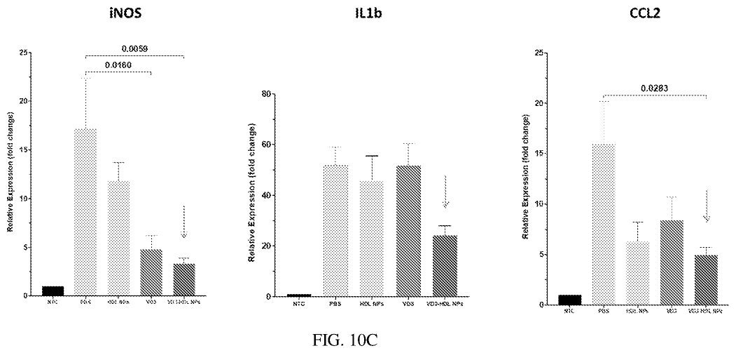

As shown in Table 1 and FIGS. 10A-10B, mice in the non-PBS treatment groups experienced statistically significant decreases in percent skin thickness relative to the PBS treatment groups for the 250 nM HDL-NP/0.19 mg Calcitriol and 750 nM HDL-NP/0.56 mg Calcitriol sets, starting at Day 3. In particular, the delivery of calcitriol using the HDL-NPs loaded with calcitriol provided significant benefit to skin thickness in these mice, an indicator of the capability of these NPs to improve wound healing and reduce inflammation.

Markers of inflammation were assessed by RT-PCR (FIG. 10C) in the mice after sacrifice on Day 5. Vitamin D3-loaded HDL NPs statistically suppressed markers of inflammation (iNOS, IL-1b, and CCL2), relative to PBS and empty HDL-NP treatments.

These data demonstrate that HDL-NPs of the disclosure can be useful in reducing inflammation and improving wound healing in inflamed and injured skin tissues.

### OTHER EMBODIMENTS AND EQUIVALENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims. While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of reducing inflammation in the eye or skin of a subject, the method comprising:
administering to the eye or skin of the subject a synthetic nanoparticle comprising a nanoparticle core and a shell comprising a lipid, wherein the shell is surrounding and attached to the nanoparticle core,
wherein the synthetic nanoparticle is administered in an effective amount to reduce inflammation in the eye or skin.

2. The method of claim 1, wherein the synthetic nanoparticle further comprises an anti-inflammatory molecule.

3. The method of claim 2, wherein the anti-inflammatory molecule is Vitamin D3.

4. The method of claim 2 or 3, wherein the anti-inflammatory molecule is associated with the core of the synthetic nanoparticle and/or the shell of the synthetic nanoparticle.

5. The method of any one of claims 2-4, wherein the anti-inflammatory molecule is attached to the synthetic nanoparticle via a covalent bond, ionic interactions, hydrophobic and/or hydrophilic interactions, electrostatic interactions, van der Waals interactions, or combinations thereof.

6. The method of any one of claims 2-5, wherein the nanoparticle comprises 10-100 anti-inflammatory molecules, optionally wherein the nanoparticle comprises about 40 anti-inflammatory molecules.

7. A method of delivering an anti-inflammatory molecule to the eye or skin of a subject, the method comprising:
administering to the eye or skin of the subject a synthetic nanoparticle comprising a nanoparticle core and a shell comprising a lipid, wherein the shell is surrounding and attached to the nanoparticle core,
wherein the synthetic nanoparticle further comprises an anti-inflammatory molecule.

8. The method of claim 7, wherein the anti-inflammatory molecule is Vitamin D3.

9. The method of claim 7 or 8, wherein the anti-inflammatory molecule is associated with the core of the synthetic nanoparticle and/or the shell of the synthetic nanoparticle.

10. The method of any one of claims 7-9, wherein the anti-inflammatory molecule is attached to the synthetic nanoparticle via a covalent bond, ionic interactions, hydrophobic and/or hydrophilic interactions, electrostatic interactions, van der Waals interactions, or combinations thereof.

11. The method of any one of claims 7-10, wherein the nanoparticle comprises 10-100 anti-inflammatory molecules, optionally wherein the nanoparticle comprises about 40 anti-inflammatory molecules.

12. A method of reducing inflammation in the eye or skin of a subject, the method comprising:
administering to the eye or skin of the subject an anti-inflammatory formulation comprising (a) a synthetic nanoparticle comprising a nanoparticle core and a shell comprising a lipid, wherein the shell is surrounding and attached to the nanoparticle core, and (b) an anti-inflammatory molecule;
wherein the anti-inflammatory formulation is administered in an effective amount to reduce inflammation in the eye or skin.

13. The method of claim 12, wherein the anti-inflammatory molecule is Vitamin D3.

14. The method of any preceding claim, wherein the administering step comprises topical administration, intraocular administration, or intradermal administration.

15. The method of any preceding claim, wherein the method results in reduction of expression of at least one inflammatory gene in the eye or skin relative to a baseline measurement.

16. The method of claim 15, wherein the at least one inflammatory gene is selected from the group consisting of: Acta2, Enos, Il1a, Inos, Tgfb, Il12r, Pdgfb, Vegfa, Cox2, Il1b, Il6, Mmp12, Mmp9, and Ccl2.

17. The method of claim 15 or 16, wherein the baseline measurement is an expression level of the eye or skin prior to the administering step, or an expression level of an untreated eye or skin.

18. The method of any preceding claim, wherein the subject is a human subject.

19. The method of any preceding claim, wherein the subject has an inflammatory disease or disorder.

20. The method of claim 19, wherein the inflammatory disease or disorder is selected from the group consisting of: corneal inflammation, corneal regeneration, ocular inflammation, age-related skin deterioration, psoriasis, atopic dermatitis, and ocular surface diseases.

21. The method of claim 20, wherein the ocular surface diseases are selected from the group consisting of: chemical and thermal injury, long-term contact lens wear, severe chronic rosacea, Stevens-Johnson syndrome, atopic keratoconjunctivitis, bacterial keratitis and graft versus host disease.

22. The method of any preceding claim, wherein the method comprises administering the synthetic nanoparticle or anti-inflammatory formulation at least once per day, at least once per week, or at least once per month.

23. The method of any preceding claim, wherein the method comprises administering the synthetic nanoparticle or anti-inflammatory formulation twice per day for three days.

24. The method of any preceding claim, wherein the core is an inorganic core, optionally wherein the inorganic core is comprised of gold (Au).

25. The method of any preceding claim, wherein the synthetic nanoparticle further comprises a protein.

26. The method of claim 25, wherein the protein is an apolipoprotein, optionally wherein the apolipoprotein is apolipoprotein A-I, apolipoprotein A-II, or apolipoprotein E.

27. The method of any preceding claim, wherein the synthetic nanoparticle further comprises a cholesterol.

28. The method of any preceding claim, wherein the shell comprises a lipid monolayer or a lipid bilayer.

29. The method of claim 28, wherein at least a portion of the lipid bilayer is covalently bound to the nanoparticle core.

30. The method of any preceding claim, wherein the nanoparticle core:
(i) has a largest cross-sectional dimension of less than or equal to about 500 nanometers (nm), less than or equal to about 250 nanometers (nm), less than or equal to about 100 nanometers (nm), less than or equal to about 75 nanometers (nm), less than or equal to about 50 nanometers (nm), less than or equal to about 30 nanometers (nm), less than or equal to about 15 nanometers (nm), less than or equal to about 10 nanometers (nm), less than or equal to about 5 nanometers (nm), less than or equal to about 3 nanometers (nm); or
(ii) has a diameter of about 5-30 nm, 5-20 nm, 5-15 nm, 5-10 nm, 8-13 nm, 8-12 nm, or 10 nm.

31. The method of any preceding claim, wherein the nanoparticle core has an aspect ratio of greater than about 1:1, 3:1, or 5:1.

32. The method of any preceding claim, wherein the lipids of the shell are phospholipids.

33. The method of claim 32, wherein the phospholipids comprise 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoethanolamine (16:0 PE), 1,2-distearoyl-sn-glycero-3-phosphoethanolamine (18:0 PE), sphingomyelin, 1,2-dioleoyl-3-trimethylammonium-propane (DOTAP), or a combination thereof.

34. The method of any preceding claim, wherein the core is an organic core.

35. The method of any preceding claim, wherein the nanoparticle further comprises a DNA molecule.

36. The method of claim 34 or 35, wherein the organic core comprises a hydrophobic phospholipid conjugated scaffold, optionally wherein the hydrophobic phospholipid conjugated scaffold is PL4.

37. The method of any preceding claim, wherein the organic core comprises an amphiphilic DNA-linked small molecule-phospholipid conjugate (DNA-PL4).

38. A synthetic nanoparticle comprising a nanoparticle core, a shell comprising a lipid surrounding and attached to the nanoparticle core, and an anti-inflammatory molecule.

39. The synthetic nanoparticle of claim 38, wherein the anti-inflammatory molecule is Vitamin D3.

40. The synthetic nanoparticle of claim 38 or 39, wherein the anti-inflammatory molecule is attached to the synthetic nanoparticle via a covalent bond, ionic interactions, hydrophobic and/or hydrophilic interactions, electrostatic interactions, van der Waals interactions, or combinations thereof.
